(19) United States
(12) Patent Application Publication
(10) Pub. No.: US 2022/0128304 A1
(43) Pub. Date: Apr. 28, 2022

(54) HEATING APPARATUS, METHOD OF USING AND MAKING THE SAME

(71) Applicant: MEDICAL CREATIONS, INC., Aspen, CO (US)

(72) Inventor: Mark Joseph, Aspen, CO (US)

(21) Appl. No.: 17/511,226

(22) Filed: Oct. 26, 2021

Related U.S. Application Data
(60) Provisional application No. 63/105,695, filed on Oct. 26, 2020.

(51) Int. Cl. F27B 17/02; F27D 7/04; H05B 3/66; H05B 1/02; F27D 5/00; A61F 2/80

(52) U.S. Cl. CPC F27B 17/02; F27D 7/04; H05B 3/66; A61F 2002/5052; F27D 5/00; A61F 2/80; F27D 2007/045; H05B 1/025

(57) ABSTRACT

The present invention generally relates to a heating apparatus, method of using and making the same that can be used to heat a thermoformable prosthetic socket.

### CROSS-REFERENCE TO RELATED APPLICATIONS

This application claims priority to and benefit under 35 U.S.C. § 119(e) of U.S. Provisional Patent Application No. 63/105,695 filed Oct. 26, 2020, the above-identified application is fully incorporated herein by reference as set forth in its entirety.

### BACKGROUND OF THE INVENTION

### Field of the Invention

The present invention generally relates to a heating apparatus, method of using and making the same, and more particularly to a heating apparatus configured to heat a prosthetic device having a thermoformable prosthetic socket to a thermoformable temperature.

### SUMMARY OF THE INVENTION

Accordingly, the invention is directed to a heating apparatus, method of using and making the same.

An advantage of the invention of heating apparatus is to differentially heat a prosthetic socket to achieve varying degrees of heating in different parts of the socket according to varied heating needs of the thermoformable material of the socket.

Another advantage of an embodiment of the heating apparatus is to keep a portion of the thermoformable socket at or near room temperature by having it extend outside the heating apparatus while the remainder of the socket is heated inside the heating apparatus to a temperature at which it becomes formable or moldable directly to a patient or directly to a model, e.g., plaster limb model of a patient.

Another advantage of an embodiment of the heating apparatus is to evenly heat the thermoformable prosthetic socket using one or more infrared heating elements that cause the heat to be driven into the depth of the socket walls.

Still yet another advantage of an embodiment of the heating apparatus is to evenly heat the thermoformable prosthetic socket using three or more separate and evenly spaced heating elements. The heating elements are controllable in an on and off fashion to allow in order mimic a rotational element by allowing the heat to rotate around a socket fixed in place.

Yet another advantage of an embodiment of the heating apparatus is to evenly heat the thermoformable prosthetic socket using a rotating apparatus configured to turn the thermoformable socket in a rotisserie like fashion in an enclosed oven to more evenly heat the socket.

Yet still another advantage of an embodiment of the invention is a heating apparatus configured to evenly heat the thermoformable prosthetic socket by using an additional infrared heating element placed inside at least a portion of the thermoformable socket so as to heat the material from both the inside and outside substantially simultaneously.

In one embodiment, a heating apparatus is configured to heat a thermoformable prosthetic socket of a prosthetic device. The heating apparatus includes a heating chamber comprising a top, a bottom and sidewalls and one or more heating elements is arranged within the heating chamber, wherein the heating chamber comprises an opening in one of the top, bottom or sidewalls. the opening is configured to allow a portion of the prosthetic device to extend outside the heating chamber when the thermoformable prosthetic is arranged within the heating chamber, so the portion extending outside the heating chamber is not heated to a thermoformable temperature when the one or more heating elements is activated.

In one embodiment, a heating apparatus is configured to heat a thermoformable prosthetic device. The heating apparatus includes a heating chamber having an enclosed bottom and a sidewall extending from the enclosed bottom and defining an open top region, a central support structure disposed within the heating chamber, the central support structure being configured to receive at least a portion of a thermoformable prosthetic socket of the thermoformable prosthetic device, and a lid configured to cover the open top region, the lid defining an opening that is configured to allow a lower portion of the thermoformable prosthetic device to extend through the opening to a region outside the heating chamber and an upper portion of the thermoformable prosthetic device configured to be arranged in the heating chamber when the at least the portion of the thermoformable prosthetic socket is engaged with the central support structure. The heating apparatus also includes one or more heating elements arranged within the heating chamber.

In one embodiment, a thermoformable prosthetic socket includes a cup shaped upper portion that is shaped to conform to an amputee's residual limb when it is heated and becomes pliable or moldable. The socket includes an open proximal end to receive a portion of the residual limb and a substantially closed distal end. The distal end of the socket is configured to connect to various prosthetic components. The prosthetic components can include metal components, adjusting systems and a prosthetic features, e.g., hand or foot features.

The distal end is configured to receive the components to fasten to, adjust to and support a prosthetic limb and as such it encounters a great deal of weight and loads. The distal end of the socket may have threaded inserts and interfacing components for attachment. The add-on components on the distal end of the thermoformable socket are precisely produced and may include threaded attachments such as screws, bolts and insert elements so that they reliably engage with the prosthetic attachment.

The distal end of the thermoformable socket is thermoformable and the components are attached to the distal socket end in close proximity to the residual limb. Accordingly, it is practical to make the distal end of the thermoformable socket out of the same thermoplastic material as the socket cup. If the socket is heated to the forming temperature in whole, the distal elements that engage with the prosthesis, screws, bolts, adjustment system and insert elements would also be thermoformable and in the process of forming the socket to the residual limb or plaster limb model, can become deformed so that they no longer engage properly. The heating apparatus is devised in such a manner so as to heat the socket cup portion and not heat the distal end components that could be altered or damaged due to heating. This can be achieved by having the distal end of the thermoformable socket that does not need to be heated extend into another area outside the heating chamber or oven. This area may be outside the heating chamber altogether or may be in a separate compartment where no heat is applied. Another challenge is to evenly heat the thermoformable socket so that all areas needing to be formed are heated to the same temperature and that the material is heated all the way through to its core so the socket can be properly formed. This can be achieved by rotating the heating element or elements activation in a circular pattern or by rotating the thermoformable socket in a rotisserie type fashion. Additionally, a heating element can be placed inside the thermoformable socket so that the socket is heated from both the inside and outside substantially simultaneously.

In one embodiment, a method of using a heating apparatus to heat a thermoformable prosthetic device. The method includes providing a heating apparatus comprising a heating chamber having an enclosed bottom and a sidewall extending from the enclosed bottom and defining an open top region; a central support structure disposed within the heating chamber; a lid configured to cover the open top region, the lid defining an opening; and one or more heating elements arranged within the heating chamber. The method also includes arranging at least a portion of a thermoformable prosthetic device over a portion of the central support structure, arranging the lid on the open top region such that a lower portion of the thermoformable prosthetic device through the opening of the lid to a region outside the heating chamber and an upper portion of the thermoformable prosthetic device is arranged in the heating chamber, and operating the heating apparatus to heat a thermoformable prosthetic socket of the thermoformable prosthetic device for a predetermined time and to a predetermined temperature.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

This Summary section is neither intended to be, nor should be, construed as being representative of the full extent and scope of the present disclosure. Additional benefits, features and embodiments of the present disclosure are set forth in the attached figures and in the description hereinbelow, and as described by the claims. Accordingly, it should be understood that this Summary section may not contain all of the aspects and embodiments claimed herein.

Additionally, the disclosure herein is not meant to be limiting or restrictive in any manner. Moreover, the present disclosure is intended to provide an understanding to those of ordinary skill in the art of one or more representative embodiments supporting the claims. Thus, it is important that the claims be regarded as having a scope including constructions of various features of the present disclosure insofar as they do not depart from the scope of the methods and apparatuses consistent with the present disclosure (including the originally filed claims). Moreover, the present disclosure is intended to encompass and include obvious improvements and modifications of the present disclosure.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 illustrates an exemplary perspective view of a heating apparatus according to an embodiment of the invention.

FIG. 2 illustrates a view of the heating apparatus according to FIG. 1 with the lid removed.

FIG. 3 illustrates an exemplary perspective view of a heating apparatus according to FIG. 1 with the lid removed and a prosthetic device arranged at least partially within the heating apparatus.

FIG. 4 illustrates an exploded enlarged view of a support for a heating apparatus according to FIG. 1.

FIG. 5 illustrates an assembled view of a support for a heating apparatus according to FIG. 1.

FIG. 6 illustrates cross-sectional view the heating apparatus with a prosthetic device according to FIG. 1.

FIG. 7 illustrates an enlarged cross-sectional view of FIG. 6.

FIG. 8 illustrates cross-sectional view the heating apparatus with a prosthetic device according to FIG. 1.

FIG. 9 illustrates an exemplary perspective view of a heating apparatus according to another embodiment of the invention.

FIG. 10 illustrates an exemplary perspective view of a heating apparatus according to FIG. 9.

FIG. 11 illustrates an exemplary perspective view of a heating apparatus according to another embodiment of the invention.

FIG. 12 illustrates an exemplary perspective view of a heating apparatus according to another embodiment of the invention.

FIG. 13 illustrates an exemplary perspective view of a heating apparatus according to another embodiment of the invention.

FIG. 14 illustrates an exemplary perspective view of a heating apparatus according to another embodiment of the invention.

FIG. 15 illustrates an exemplary perspective view of a heating apparatus according to FIG. 14.

FIG. 16 illustrates an exemplary perspective view of a heating apparatus according to another embodiment of the invention.

FIG. 17 illustrates an exemplary perspective view of a heating apparatus according to another embodiment of the invention.

FIG. 18 illustrates a method of using a heating apparatus to heat a prosthetic device according to another embodiment of the invention.

### DETAILED DESCRIPTION OF THE ILLUSTRATED EMBODIMENTS

The following detailed description describes a heating apparatus, method of using and making the same and is presented to enable any person skilled in the art to make and use the disclosed subject matter in the context of one or more particular implementations. Various modifications, alterations, and permutations of the disclosed implementations can be made and will be readily apparent to those skilled in the art, and the general principles defined may be applied to other implementations and applications, without departing from scope of the disclosure. The present disclosure is not intended to be limited to the described or illustrated implementations, but to be accorded the widest scope consistent with the described principles and features.

In order to more fully appreciate the present disclosure and to provide additional related features, each of the following references are fully incorporated therein by reference in their entireties:

In one embodiment, a prosthetic socket includes a conical cup, upper portion or called socket herein, base (lower portion) and attachment mechanisms as described with U.S. Pat. Nos. 15/914,480, 16/516,199, 16/897,425, and 17/001,380 each application is hereby incorporated by reference as if fully set forth herein. Each of these heating systems can be used with any prosthetic socket.

In one embodiment, the prosthetic socket includes a conical cup (upper portion) and base (lower portion) can include a thermoformable material includes one or more of a thermoplastic elastomer material, polyester blend, polyester material, a thermoplastic polyurethane (TPU) material, a polycaprolactone material or blend thereof, an ABS material or blend thereof, a polypropylene material or blend thereof, A PVC material or blend thereof, a polyethylene material or blend thereof, or other polymer materials or blends considered to be thermo formable in a temperature range of 160° F. to 325° F. Optionally, additives can be added, e.g., the additives can be incorporated into the higher temperature thermoplastic to improve strength including but not limited to carbon fiber, aramid fiber, fiberglass, glass micro, carbon nanotubes beads, and others.

The conical cup includes a material having a first pliability at between about 160° F. and about 302° F. (between about 70° C. and about 150° C.) to be stretched circumferentially over a residual limb, a lower portion coupled to a lower surface of the conical cup creating an enclosed form having a second pliability which is less than the first pliability, and a base coupled to the lower portion, wherein the conical cup and the lower portion are injection molded of a thermoplastic polymer, wherein the conical cup and the lower portion, when heated to between about 160° F. and about 302° F. (between about 70° C. and about 150° C.) have a working time of between about five minutes and about 15 minutes before hardening as room temperature is approached, and wherein the conical cup and the lower portion each comprise a hardness exceeding ASTM D2240 of 50 D shore hardness, a tensile strength exceeding ASTM D638 of 5,000 psi, and a flexural modulus exceeding ASTM D5023 of 150,000 PSI.

The conical cup and the lower portion can be unitary system or material. Further, at least one of the conical cup and the lower portion can be injection molded, and the other of the conical cup and the lower is injection over-molded. The thermoplastic polymer of the conical cup and base can comprise at least one additive from the group of fiberglass, carbon fiber, aramid fiber, glass beads, and carbon nanotubes. The conical cup and lower portion can be made from the same or different materials.

Reference will now be made in detail to an embodiment of the present invention, example of which is illustrated in the accompanying drawings.

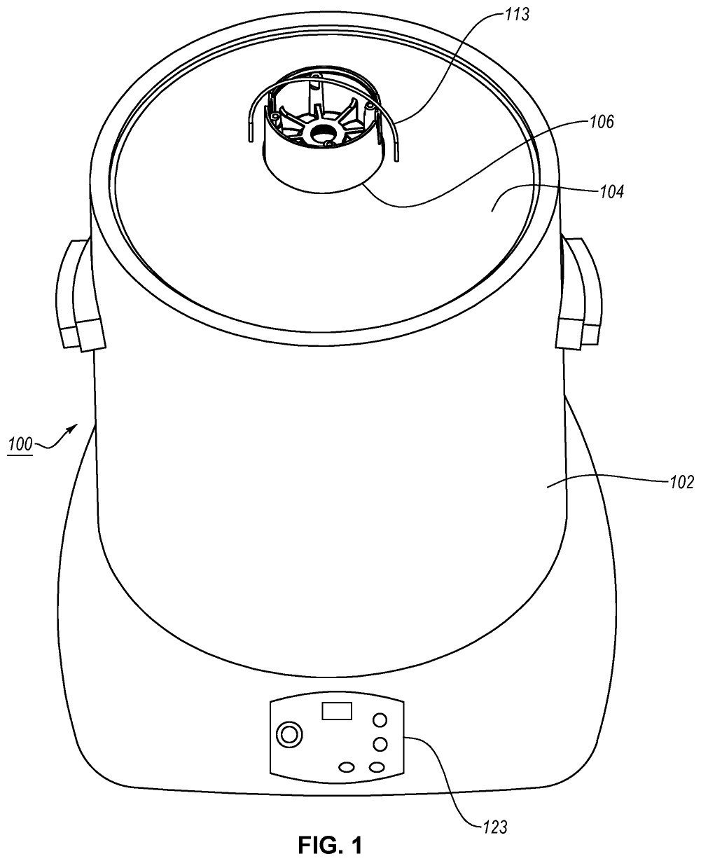

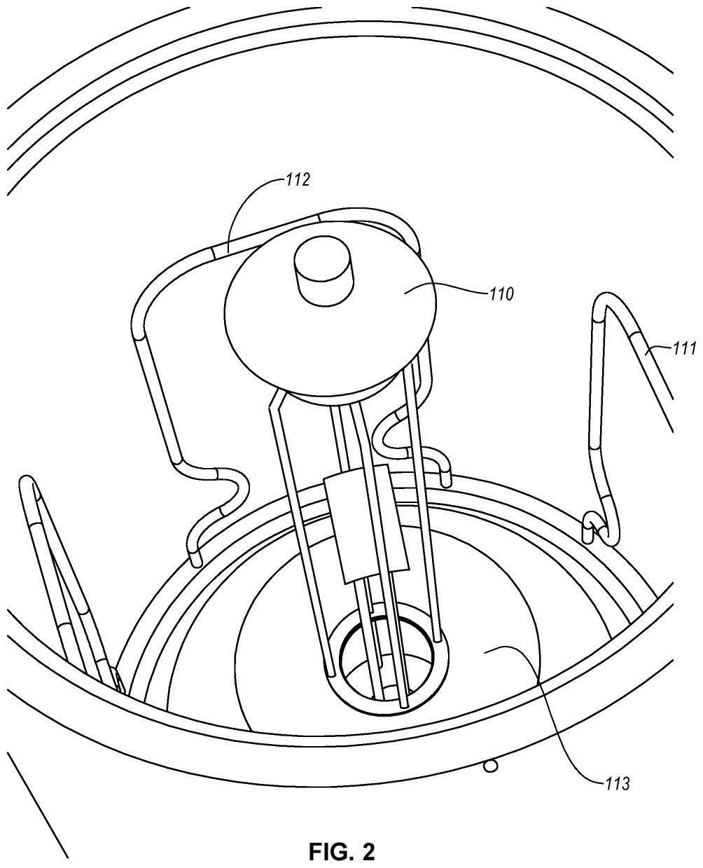

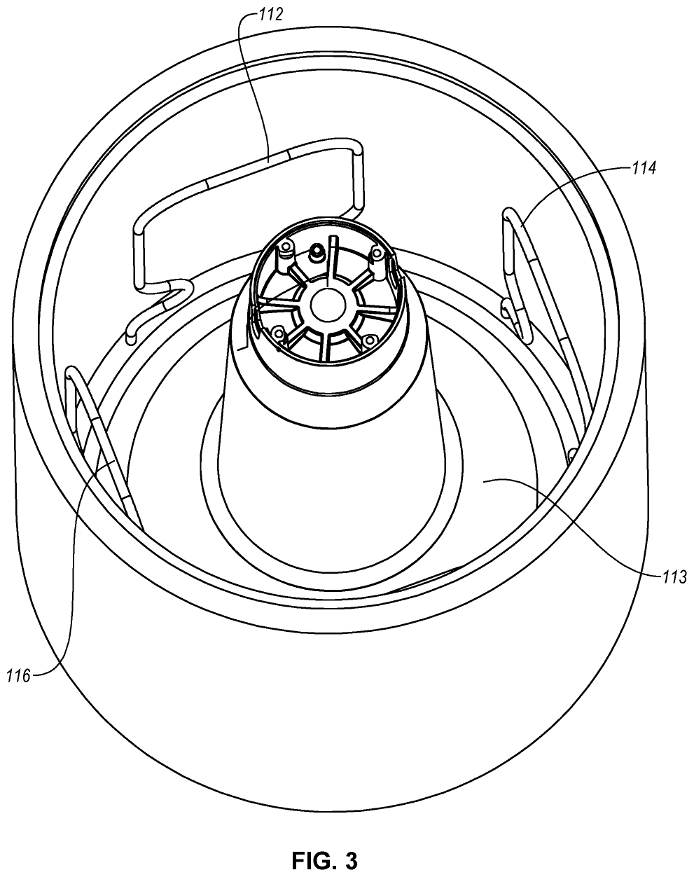

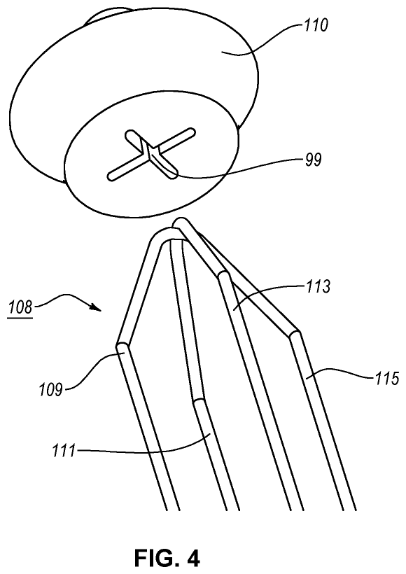

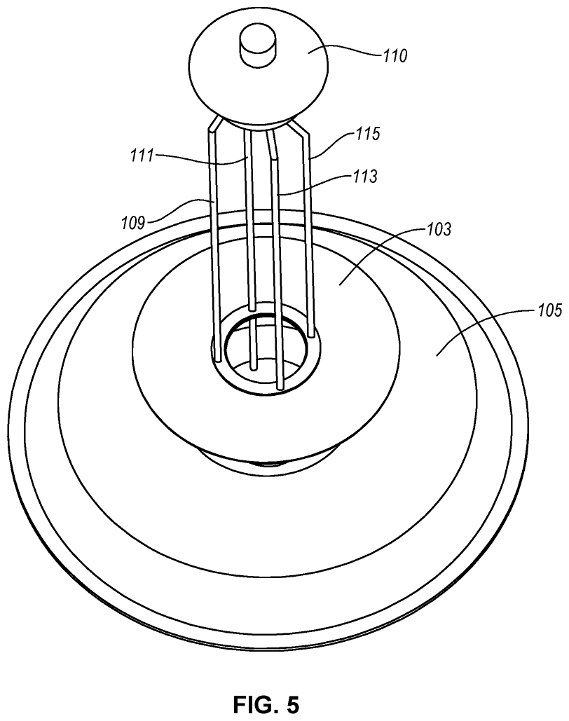

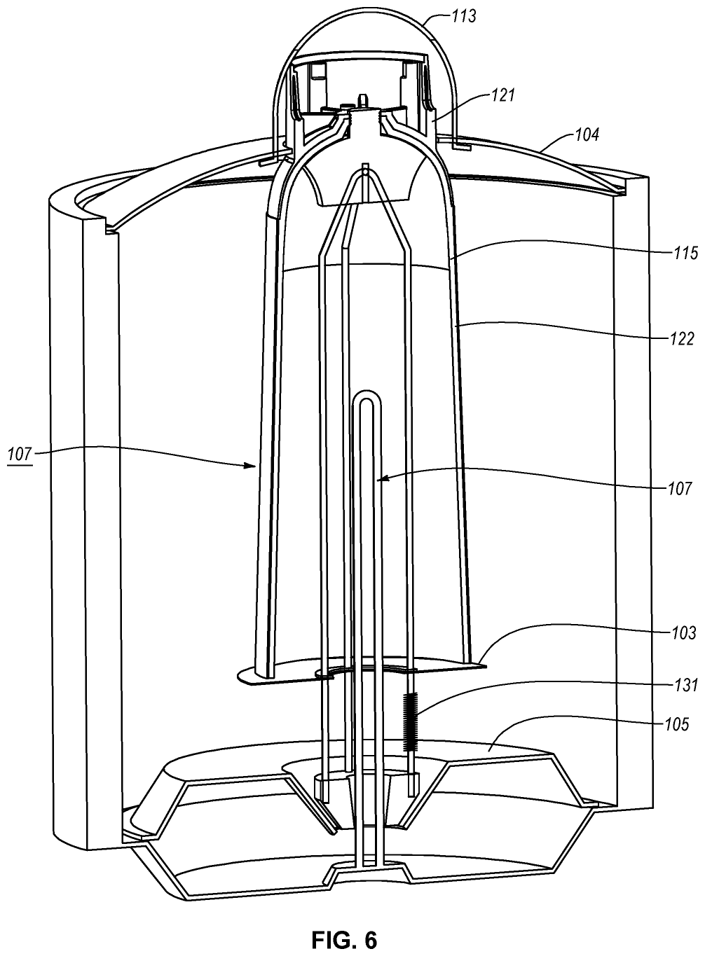

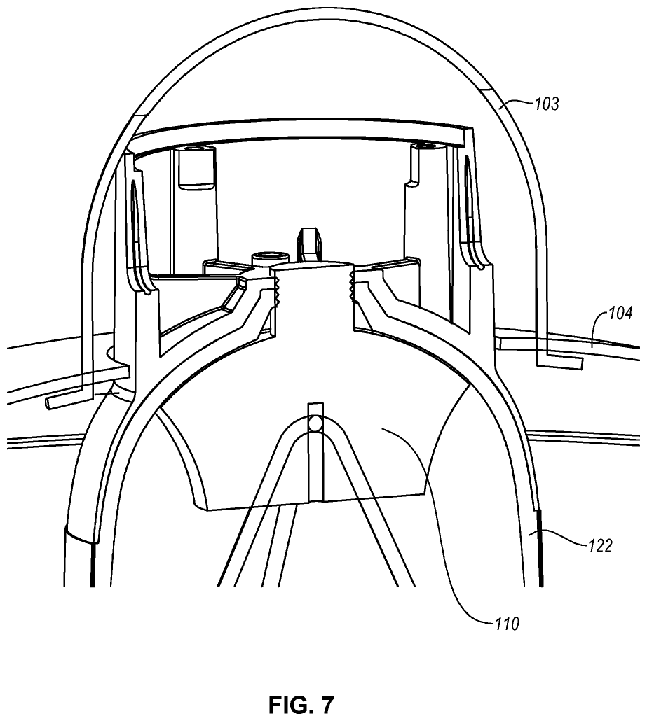

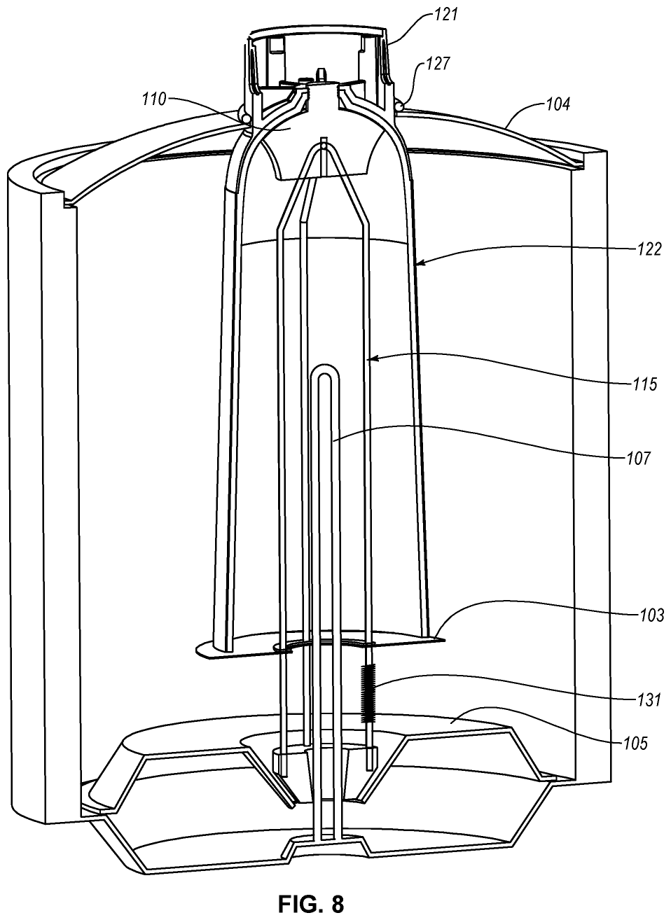

FIG. 1 illustrates an exemplary perspective view of a heating apparatus according to an embodiment of the invention. FIG. 2 illustrates a view of the heating apparatus according to FIG. 1 with the lid removed. FIG. 3 illustrates an exemplary perspective view of a heating apparatus according to FIG. 1 with the lid removed and a prosthetic device arranged at least partially within the heating apparatus. FIG. 4 illustrates an exploded enlarged view of a support for a heating apparatus according to FIG. 1. FIG. 5 illustrates an assembled view of a support for a heating apparatus according to FIG. 1. FIG. 6 illustrates cross-sectional view the heating apparatus with a prosthetic device according to FIG. 1. FIG. 7 illustrates an enlarged cross-sectional view of FIG. 6. FIG. 8 illustrates cross-sectional view the heating apparatus with a prosthetic device according to FIG. 1.

Referring to FIGS. 1-8, the invention generally relates to a heating apparatus 100 is used to heat a thermoformable prosthetic socket of a prosthetic socket to a predetermined temperature for a predetermined time. In one embodiment, the predetermined temperature is to a temperature where the thermoformable prosthetic socket becomes thermoformable. In one embodiment, thermoformable is where the thermoformable prosthetic socket becomes malleable and moldable. In one embodiment, the predetermined temperature is in a range from about 160° F. to about 302° F. (between about 70 C and about 150 C).

At this thermoformable temperature the thermoformable prosthetic socket can be stretched circumferentially over a residual limb or a molder of a user's residual's limb. In a preferred embodiment, the model has been globally reduced. Global reduction is the process of hand filing, scraping and sanding of the plaster model to reduce the circumference in a global manner to make the resulting prosthetic socket more tightly fit the residual limb so as to bear weight in a more global manner reducing weight born by the residual bone end. In one embodiment, the prosthetic socket is described U.S. patent application Ser. Nos. 15/914,480, 16/516,199, 16/897,425, and 17/001,380, each of which are hereby incorporated by reference as if fully set forth herein. The prosthetic socket can be formed and molded onto a residual limb or model as described with U.S. patent application Ser. Nos. 15/914,480, 16/516,199, 16/897,425, and 17/001,380, each of which are hereby incorporated by reference.

In one embodiment, the prosthetic socket also includes a rigid region configured to receive interchangeable suspension system and mechanical parts. The material of the rigid region can be same or different from the thermoformable prosthetic socket.

In this embodiment, the apparatus 100 is configured to heat the thermoformable prosthetic socket to a thermoformable temperature and not heat the rigid portion of the prosthetic device to a thermoformable temperature. Heating other portions of the prosthetic device above a critical temperature, e.g., 140 F can cause problems with material of the rigid portion, i.e., base portion, e.g., distorting it, weakening it, bonding problems between the base portion and the upper portion. The current design assures that there is plenty of surface for the over molding to chemically bond to the inserted base cup.

In a preferred embodiment, the thermoformable prosthetic socket should not be exposed to oven temperatures greater than about 280° F. or other energy sources as higher temperatures could cause surface temperatures of the upper socket to exceed 280° F. Exposure to temperatures between about 140° F. to about 280° F. should not exceed thirty minutes. Additionally, the lower portion or rigid portion of the prosthetic socket base should not be exposed to temperatures of greater than about 130° F. For this reason, the Below knee prosthetic should only be heated with the oven and other approved devices, as described in the product manual.

In this embodiment, the heating apparatus 100 includes a body region 102, a lid 104, and a handle 113 coupled to the lid. The lid 104 includes an opening 106 that allows the base portion or lower portion of the prosthetic socket to not be heated to the thermoformable temperature as it arranged outside the heating area that occurs inside a heating chamber of the apparatus 100. That is, heating only occurs in the desired areas of the heating apparatus 100. Inside the heating apparatus is a heating chamber 105 that includes a support mechanism 108 including four supports that is configured to suspend at least a portion of the prosthetic socket and support pedestal 110 arranged and attached to each of the supports.

A heating element 107 is arranged inside the support mechanism 108. The heating element 107 is configured to heat an interior portion of the prosthetic socket. Optionally and/or alternatively, an adjustable heat shield 109 is utilized to vary the amount of heat emitted from the heating element 107. The adjustable heat shield 109 can be expanded to cover a portion of the heating element 107.

The support mechanism 108 includes four supports, a first support 109, a second support 111, a third support 113 and a fourth support 115, however, any number of supports may be utilized. Each of the supports are configured to be arranged in a recess 99 of the support pedestal 110. The supports are arranged through a heat shield 103 and heat shield 105 resides in the apparatus as well as shown in including an upper support pedestal 110 and a lower support plate 103 that are configured to suspend and support the socket as it is heated and becomes pliable. Heat shield 103 is supported by one or more expandable members 111, e.g., springs, configured to support the heat shield a predetermined vertical orientation.

An upper support pedestal 110 is arranged and attached to support legs 108. Any number of support legs may be utilized, e.g., four are shown in this embodiment. The upper support pedestal 110 attaches to the support legs 108 by inserting into recesses 117. The support legs pass through the lower support plate 103. In this embodiment, the support plate 103 has a heat proof covering of nonstick material such as Teflon 105 as shown in FIG. 8.

To properly fit the socket length, the support plate 103 can be adjusted in height by means of a locking apparatus on its supports or it can be mounted on rods with springs 111 to support the plate 103 so as to automatically support the proximal end of the prosthetic socket 117 properly. A heating element 107 is arranged inside the support legs 108 to heat the inside of the socket. A number of heating elements 112, 114, and 116 are also arranged around the inside perimeter of the heating apparatus as shown in FIG. 3. In this embodiment, the prosthetic socket 117 includes an upper region or socket region as a thermoformable socket 121 and a base region 122.

In one embodiment, the heating apparatus 100 has controls to control the different heating elements 107, 112, 114 and 116, at various temperatures and different timings. The heating elements 107, 112, 114, and 116 can be configured to time on and off in a rotating pattern to simulate a rotisserie effect to more evenly heat the socket. Optionally, the heating elements 107, 112, 114, and 116 can be configured to time on and off in a constant fashion.

Optionally and/or alternatively, the one or more heating elements are configured to work with a controller to and thermocouple 113. In one embodiment, the controller is configured to turn one and off the different heating elements at a predetermined set temperature or predetermined set time. For example, a user can set a desired temperature and/or desired heating time and after either or both have been established the heating elements are turned off.

In embodiments, the heating elements 107, 112, 114 and 116 are infrared heating elements. There are a number of ways to heat the prosthetic socket, e.g., with infrared electric heating elements that use induction like in a typical electric cooking oven or a BBQ lighter. It is believed infrared heating allows for rapid heating of the thermoformable prosthetic socket 120.

In one embodiment, in operation, infrared heating radiates out from each of the heating elements 107, 112, 114 and 116. The system is configured to allow the socket 120 to rotate with a rotating mechanism via a pedestal 108. Optionally or alternatively, three heating elements can also be configured, e.g., separately wired, to control each heating element, e.g., operating in an on and off rotating fashion as well as a center element to mimic rotation.

As described herein, prosthetic socket 117 includes a thermoformable socket 121 and rigid base portion 122. It is also believed that heating the prosthetic socket 117 from the inside aided in getting heat into the core of the polymer quicker and more evenly, e.g., with heating element 107. In one embodiment, the lid 104 includes a hole 106 that allows the base portion 122 of the prosthetic socket 117 to protrude and not directly heated by heating apparatus 100. The thermoformable socket 121 region is inside the heat chamber of the apparatus 100.

In this embodiment, the pedestal 108 is a center armature that supports the prosthetic socket 117. The support pedestal 110 is insulated, e.g., made from a silicone material, to insulate the base cup 122 from getting heated. It has an armature to support this and prevent the socket from touching the inner heating element. A suspended spring 111 mounted base plate 103 with Teflon ring is configured to support the proximal end of the prosthetic socket 107. If not supported from both ends the thermoformable prosthetic socket 122 can sag when heated to the thermoformable temperature.

The heating apparatus is controlled from a control panel 123 that can designate ON/OFF, the heating time, the desired end temperature and/or the function of the heating elements. In this embodiment, in operation, the infrared rays are configured to radiate out from each of the heating elements onto the socket. Optionally, and to avoid any overheating the surface of the socket in certain areas the socket 120 can rotate on a rotating support platform and pedestal.

Optionally and/or alternatively, a seal member 127 can be utilized to seal the base 121 in the lid opening 106. The seal member can be made of insulative material, e.g., silicone. Typically, the seal member 127 is utilized when the base 121 has a much smaller outer circumference.

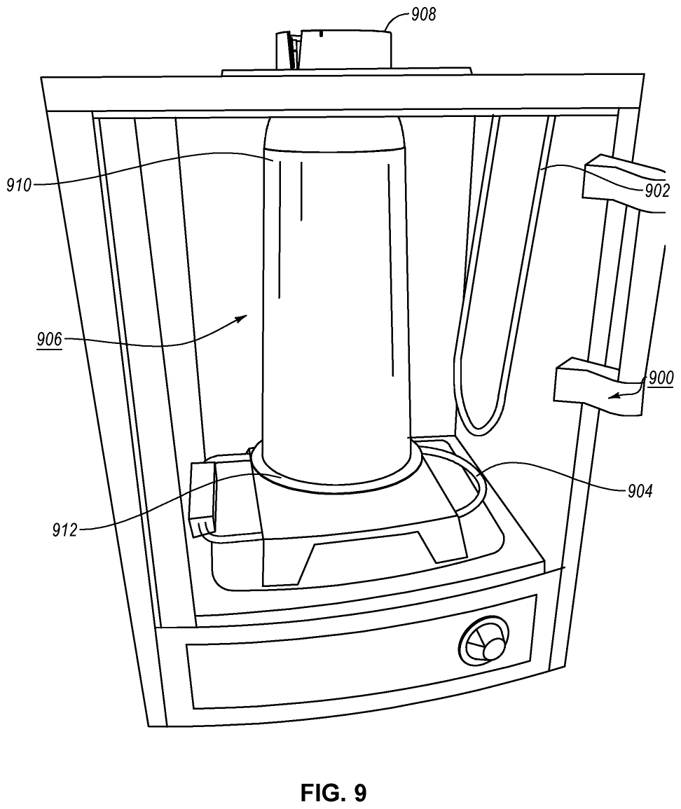

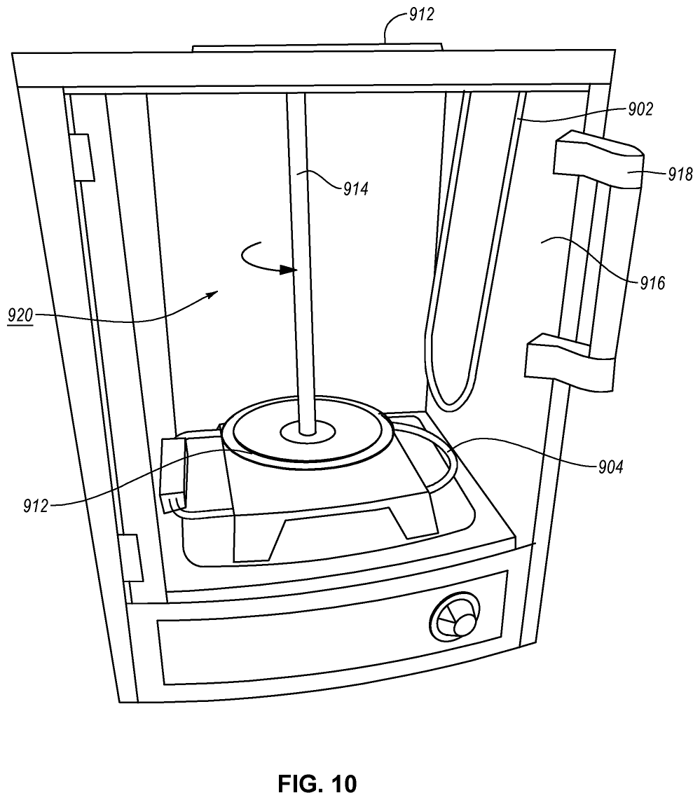

FIG. 9 illustrates an exemplary perspective view of a heating apparatus according to another embodiment of the invention. FIG. 10 illustrates an exemplary perspective view of a heating apparatus according to FIG. 9.

Referring to FIGS. 9-10, the heating apparatus 900 includes a first heating element 902 and a second heating element 904 configured to heat a prosthetic socket 906. The prosthetic socket 906 includes a base region 908 and a thermoformable socket 910. The heating apparatus 900 includes a hole 912 that is sized to allow the base region 908 to extend outside the heating apparatus 900. The hole 912 optionally includes a lid to cover the hole. The heating apparatus 900 also includes a base plate 912 and bar 914. The base plate 912 and bar 914 are configured to rotate during heating to provide more uniform heating and avoid prolonged exposure to the heating element 902. The rotating is done with a rotating with a rotating motor. The heating apparatus 900 includes a door 916 with a handle 918 to allow access to the heating chamber 920.

In one embodiment, the heating apparatus is controlled from a control panel 922 that can designate ON/OFF, the heating time, the temperature or the function of the heating elements.

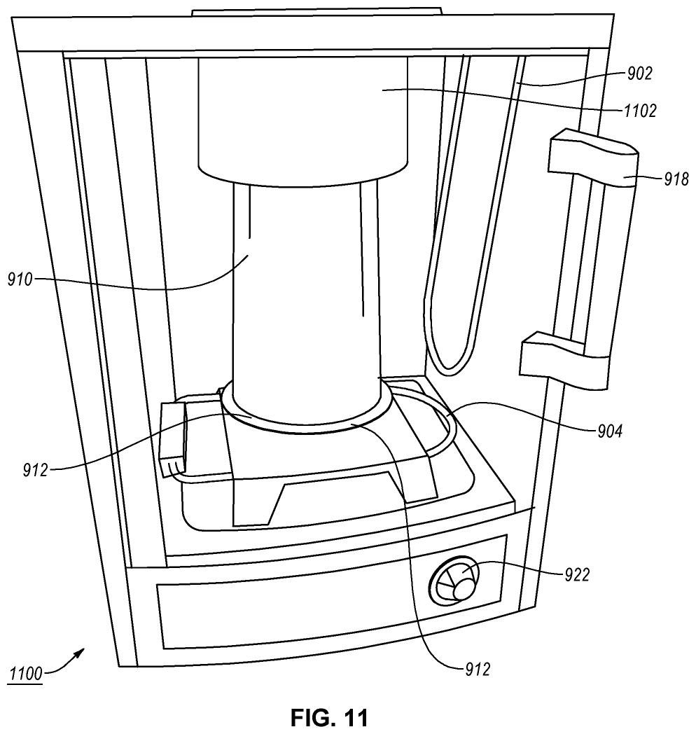

FIG. 11 illustrates an exemplary perspective view of a heating apparatus according to another embodiment of the invention.

Referring to FIG. 11, the heating apparatus 1100 includes a first heating element 902 and a second heating element 904 configured to heat a prosthetic socket 906. The prosthetic socket 906 includes a base region 908 and a thermoformable socket 910. The heating apparatus 1100 is sized to fit the entire prosthetic socket 906, without a hole 912 in FIG. 9. A thermally insulative material 1102, e.g., silicon, foil and other materials as known in the art, is arranged around a base region 908 of the prosthetic socket 906. The heating apparatus 900 also includes a base plate 912 and bar 914. The base plate 912 and bar 914 are configured to rotate during heating to provide more uniform heating and avoid prolonged exposure to the heating element 902. The rotating is done with a rotating with a rotating motor. The heating apparatus 900 includes a door 916 with a handle 918 to allow access to the heating chamber 920.

In one embodiment, the heating apparatus is controlled from a control panel 922 that can designate ON/OFF, the heating time, the temperature or the function of the heating elements.

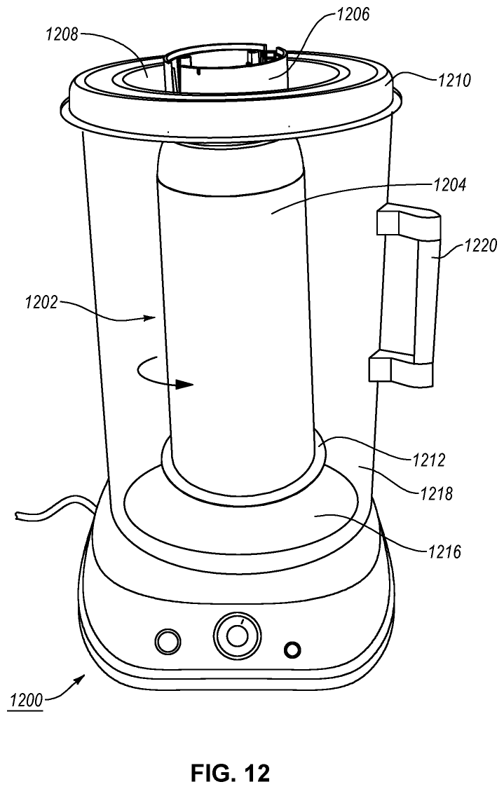

FIG. 12 illustrates an exemplary perspective view of a heating apparatus according to another embodiment of the invention.

Referring to FIG. 12, the heating apparatus 1200 includes one or more heating elements, e.g., infrared heating elements configured to heat a prosthetic socket 1202 to a thermoformable temperature. The prosthetic socket 1202 includes a base region 1204 and a thermoformable socket 1206. The heating apparatus 1200 includes a hole 1208 in a lid 1210 that is sized to allow the base region 1206 to extend outside the heating apparatus 1200. The heating apparatus 1200 also includes a base plate 1212. The base plate 1212 can be configured to rotate as indicated by arrow 1214 during heating to provide more uniform heating and avoid prolonged exposure to the one or more heating elements. A heat shield 1216 is also present. The rotating is done with a rotating with a rotating motor. The heating apparatus 1200 includes a door 1218 with a handle 1220 to allow access to the heating chamber 920.

In one embodiment, the heating apparatus is controlled from a control panel 1222 that can designate ON/OFF, the heating time, the temperature or the function of the heating elements.

The base 122 of the socket 120 extends out the apparatus 1400 through lid with a hole. The base 122 of the socket 120 extends out the apparatus 1400 through lid with a hole. The apparatus heats the socket with a heating element placed inside the socket and the close glass cylinder 1402 insulates the socket so it heats faster. The heating apparatus is controlled from a control panel 1404 that can designate ON/OFF, the heating time, the temperature or the function of the heating elements.

Referring to FIG. 12, the heating apparatus 1200 includes one or more heating elements, e.g., infrared heating elements configured to heat a prosthetic socket 1202 to a thermoformable temperature. The prosthetic socket 1202 includes a base region 1204 and a thermoformable socket 1206. The heating apparatus 1200 includes a hole 1208 in a lid 1210 that is sized to allow the base region 1206 to extend outside the heating apparatus 1200. The heating apparatus 1200 also includes a base plate 1212. The base plate 1212 can be configured to rotate as indicated by arrow 1214 during heating to provide more uniform heating and avoid prolonged exposure to the one or more heating elements. A heat shield 1216 is also present. The rotating is done with a rotating with a rotating motor. The heating apparatus 1200 includes a door 1218 with a handle 1220 to allow access to the heating chamber 920.

In one embodiment, the heating apparatus is controlled from a control panel 1222 that can designate ON/OFF, the heating time, the temperature or the function of the heating elements.

The base 122 of the socket 120 extends out the apparatus 1400 through lid with a hole. The base 122 of the socket 120 extends out the apparatus 1400 through lid with a hole. The apparatus heats the socket with a heating element placed inside the socket and the close glass cylinder 1402 insulates the socket so it heats faster. The heating apparatus is controlled from a control panel 1404 that can designate ON/OFF, the heating time, the temperature or the function of the heating elements.

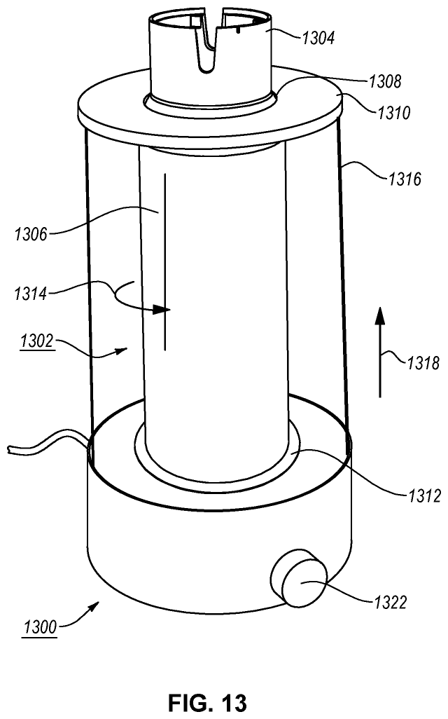

FIG. 13 illustrates an exemplary perspective view of a heating apparatus according to another embodiment of the invention.

Referring to FIG. 13, the heating apparatus 1300 includes one or more heating elements, e.g., infrared heating elements configured to heat a prosthetic socket 1302 to a thermoformable temperature. The prosthetic socket 1302 includes a base region 1304 and a thermoformable socket 1306. The heating apparatus 1300 includes a hole 1308 in a lid 1310 that is sized to allow the base region 1304 to extend outside the heating apparatus 1300. The heating apparatus 1300 also includes a base plate 1312. The base plate 1312 can be configured to rotate as indicated by arrow 1314 during heating to provide more uniform heating and avoid prolonged exposure to the one or more heating elements. A heat shield may also be present. The rotating is done with a rotating with a rotating motor. The heating apparatus 1300 is accessible by moving the outer portion 1316 upward in a direction indicated by arrow 1318.

In one embodiment, the heating apparatus 1300 is controlled from a control panel 1322 that can designate ON/OFF, the heating time, the temperature or the function of the heating elements.

The base 122 of the socket 120 extends out the apparatus 1400 through lid with a hole. The base 122 of the socket 120 extends out the apparatus 1400 through lid with a hole. The apparatus heats the socket with a heating element placed inside the socket and the close glass cylinder 1402 insulates the socket so it heats faster. The heating apparatus is controlled from a control panel 1404 that can designate ON/OFF, the heating time, the temperature or the function of the heating elements.

Referring to FIG. 12, the heating apparatus 1200 includes one or more heating elements, e.g., infrared heating elements configured to heat a prosthetic socket 1202 to a thermoformable temperature. The prosthetic socket 1202 includes a base region 1204 and a thermoformable socket 1206. The heating apparatus 1200 includes a hole 1208 in a lid 1210 that is sized to allow the base region 1206 to extend outside the heating apparatus 1200. The heating apparatus 1200 also includes a base plate 1212. The base plate 1212 can be configured to rotate as indicated by arrow 1214 during heating to provide more uniform heating and avoid prolonged exposure to the one or more heating elements. A heat shield 1216 is also present. The rotating is done with a rotating with a rotating motor. The heating apparatus 1200 includes a door 1218 with a handle 1220 to allow access to the heating chamber 920.

In one embodiment, the heating apparatus is controlled from a control panel 1222 that can designate ON/OFF, the heating time, the temperature or the function of the heating elements.

The base 122 of the socket 120 extends out the apparatus 1400 through lid with a hole. The base 122 of the socket 120 extends out the apparatus 1400 through lid with a hole. The apparatus heats the socket with a heating element placed inside the socket and the close glass cylinder 1402 insulates the socket so it heats faster. The heating apparatus is controlled from a control panel 1404 that can designate ON/OFF, the heating time, the temperature or the function of the heating elements.

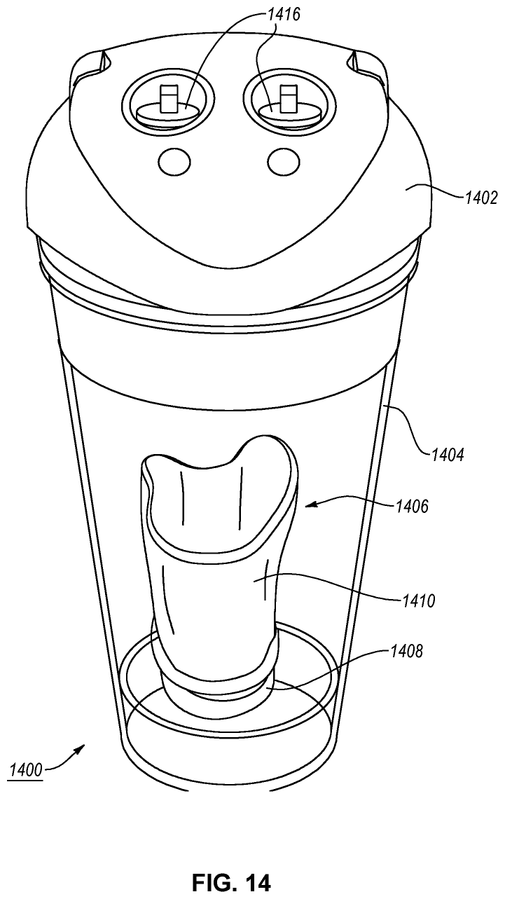

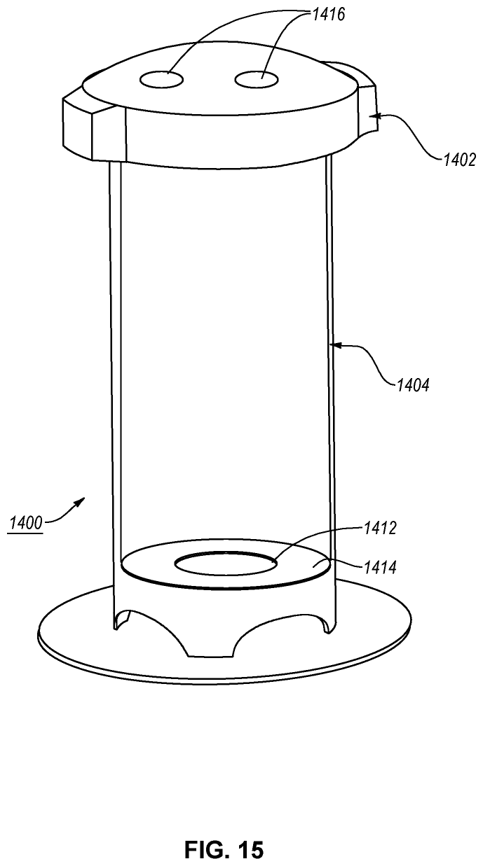

FIG. 14 illustrates an exemplary perspective view of a heating apparatus according to another embodiment of the invention. FIG. 15 illustrates an exemplary perspective view of a heating apparatus according to FIG. 14.

Referring to FIGS. 14-15, the heating apparatus 1400 includes one or more heating elements arranged in an upper portion 1402. The upper portion 1402 includes a heater, e.g., a halogen heater or an infrared heater ring inside the upper portion. The halogen heater can be configured to transmit heat. An internal fan is also position inside the upper portion to circulate the heat emitted from the heater inside the chamber 1404 in a convective manner.

The prosthetic socket 1406 includes a base region 1408 and a thermoformable socket 1410. An end portion or base 1404 of the socket can extend outside the heater chamber with a hole 1412 in the bottom or base 1414 of the heating chamber 1404. and be held in a cup or ring 122 so that it does not get heated. Optionally and/or alternatively, an insulating material can be arranged over the base 1404 to further prevent or mitigate heating.

The heating apparatus is controlled from a control panel 1416 that can designate ON/OFF, the heating time, the temperature or the function of the heating elements.

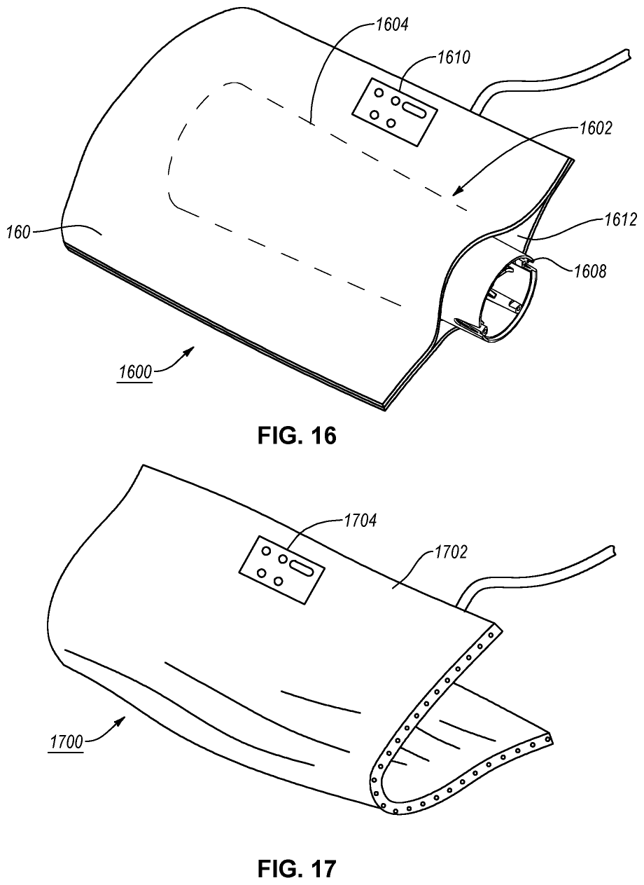

FIG. 16 illustrates an exemplary perspective view of a heating apparatus according to another embodiment of the invention.

Referring to FIG. 16, the heating apparatus 1600 includes one or more heating pads 1601. The prosthetic socket 1602 includes a base region 1608 and a thermoformable socket 1604 region. The heating pad 1601 includes a first silicone heating pad, a second silicone heating pad 1906 in communication with a controller 1610. The heating apparatus can be a heating bag or alternately can be wrapped around the region 1604 to heat to the desired temperature. A hole 1612 is utilized to have base portion 1608 extend outside the heating apparatus. The hole 1612 can be adjusted with an adjustment mechanism, e.g., zipper, Velcro, snaps or combinations of the same.

FIG. 17 illustrates an exemplary perspective view of a heating apparatus according to another embodiment of the invention.

Referring to FIG. 17, the heating apparatus 1700 includes one or more heating pads 1702. The prosthetic socket can be arranged in a folded heating pad 1702. The heating pad 1702 can be a heating bag or alternately can be wrapped around a desired region of a prosthetic socket to be heated to the desired temperature for a desired time. A controller 1704 controls the heating.

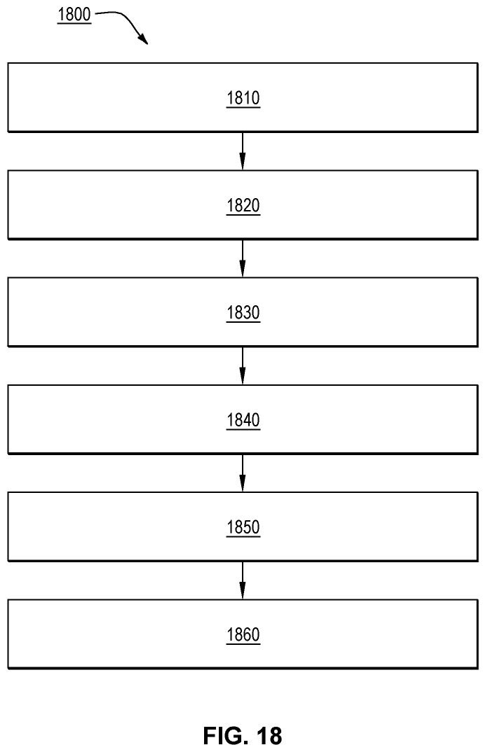

FIG. 18 illustrates a method of using a heating apparatus to heat a prosthetic device according to another embodiment of the invention.

In step 1810 a prosthetic device is placed into a heating apparatus described herein. In step 1820 the oven is turned on.

In step 1830 the prosthetic device is heated at a predetermined temperature for a predetermined time.

In step 1840 the oven is turned off

In step 1850 the heated prosthetic device is removed from the oven.

In step 1860 the heated prosthetic device is molded directly to a patient's residual limb or a model.

The inventions and methods described herein can be viewed as a whole, or as a number of separate inventions, that can be used independently or mixed and matched as desired. All inventions, steps, processed, devices, and methods described herein can be mixed and matched as desired. All previously described features, functions, or inventions described herein or by reference may be mixed and matched as desired.

It will be apparent to those skilled in the art that various modifications and variation can be made in the present invention without departing from the spirit or scope of the invention. Thus, it is intended that the present invention cover the modifications and variations of this invention provided they come within the scope of the appended claims and their equivalents.

What is claimed is:

1. A heating apparatus configured to heat a thermoformable prosthetic socket of a prosthetic device, comprising:
a heating chamber comprising a top, a bottom and sidewalls;
one or more heating elements is arranged within the heating chamber, wherein the heating chamber comprises an opening in one of the top, bottom or sidewalls, and
wherein the opening is configured to allow a portion of the prosthetic device to extend outside the heating chamber when the thermoformable prosthetic is arranged within the heating chamber, so the portion extending outside the heating chamber is not heated to a thermoformable temperature when the one or more heating elements is activated.

2. The heating apparatus of claim 1, further comprising a support structure arranged within the heating chamber, and
wherein the thermoformable prosthetic socket is configured to be supported by at least a portion of the support structure and wherein the support structure is configured to prevent the supported thermoformable prosthetic socket from substantially deforming from the thermoformable prosthetic socket shape that is not heated to a thermoformable temperature.

3. The heating apparatus of claim 2, wherein a portion of the thermoformable prosthetic socket is configured to rest upon the support structure, and
wherein the support structure is configured rotate about a central axis of the support structure.

4. The heating apparatus of claim 2, wherein the one or more heating elements comprises two or more heating elements,
wherein the thermoformable prosthetic socket is configured to rest upon the support structure in a fixed orientation, and
wherein the support structure and the two or more heating elements are configured to turn on and off in a rotational manner.

5. The heating apparatus of claim 1, wherein the one more heating elements comprises infrared heating elements.

6. The heating apparatus of claim 5, further comprises one or more fans configured for convention heating.

7. The heating apparatus of claim 1, wherein a surface of the heating chamber is configured as a removable lid and when the lid is removed it configured to access an interior of the heating chamber, and
wherein the removable lid includes an opening configured to allow a portion of the prosthetic device to extend outside the heating chamber when a portion of the prosthetic device is arranged in the heating chamber such that the thermoformable prosthetic socket is arranged in the heating chamber.

8. The heating apparatus of claim 1, wherein heating chamber comprises a non-rigid material.

9. The heating apparatus of claim 8, further comprising a mechanism configured to open and close a portion of the heating chamber in order to provide access to an interior region of the heating chamber.

10. The heating apparatus of claim 8, further comprising a zipper configured to open and close a portion of the heating chamber in order to provide access to an interior region of the heating chamber.

11. The heating apparatus of claim 8, wherein said opening of the heating chamber further comprises a non-rigid material configured to provide a seal between the prosthetic device and the non-rigid material.

12. The heating apparatus of claim 1, wherein the one or more heating elements comprises four heating elements.

13. The heating apparatus of claim 1, wherein the one or more heating elements comprises one heating element configured to be arranged within an interior portion of the thermoformable prosthetic socket.

14. A heating apparatus configured to heat a thermoformable prosthetic device, comprising:
a heating chamber having an enclosed bottom and a sidewall extending from the enclosed bottom and defining an open top region;
a central support structure disposed within the heating chamber, the central support structure being configured to receive at least a portion of a thermoformable prosthetic socket of the thermoformable prosthetic device;
a lid configured to cover the open top region, the lid defining an opening that is configured to allow a lower portion of the thermoformable prosthetic device to extend through the opening to a region outside the heating chamber and an upper portion of the thermoformable prosthetic device configured to be arranged in the heating chamber when the at least the portion of the thermoformable prosthetic socket is engaged with the central support structure; and
one or more heating elements arranged within the heating chamber.

15. The heating apparatus of claim 14, wherein a position of the central support structure is adjustable within the heating chamber.

16. The heating apparatus of claim 14, wherein central support structure further comprises a second lower support platform configured to physically support a proximal end of the upper portion of the thermoformable prosthetic device when the at least the portion of the thermoformable prosthetic socket is engaged with the central support structure.

17. The heating apparatus of claim 16, wherein a position of the second lower support platform within the heating chamber is adjustable with respect to a proximal end of the central support structure.

18. The heating apparatus of claim 14, wherein the one or more heating elements comprises a first heating element arranged adjacent to the sidewall, a second heating element arranged adjacent to the circular sidewall, and a third heating element arranged adjacent to the circular sidewall, wherein the first heating element, second heating element and the third heating element are substantially equally spaced apart radially around the circular sidewall.

19. The heating apparatus of claim 18, further comprising a controller configured to selectively activate each one of the first, second, and third heating elements.

20. The heating apparatus of claim 18, further comprising a fourth heating element disposed within the central support.
